(19) United States
(12) Patent Application Publication
(10) Pub. No.: US 2025/0092093 A1
(43) Pub. Date: Mar. 20, 2025

(54) Androgen receptor protein degradation-inducing peptides for prostate cancer treatment

(71) Applicant: Benyi Li, OVERLAND PARK, KS (US)

(72) Inventor: Benyi Li, OVERLAND PARK, KS (US)

(21) Appl. No.: 18/470,073

(22) Filed: Dec. 31, 2023

(30) Foreign Application Priority Data
Sep. 19, 2023 (AU) 18470073
Sep. 19, 2023 (CA) 18470073
Sep. 19, 2023 (CN) 18470073
Sep. 19, 2023 (EM) 18470073
Sep. 19, 2023 (GC) 18470073
Sep. 19, 2023 (JP) 18470073

(51) Int. Cl. C07K 7/64; A61K 38/00

(52) U.S. Cl. CPC C07K 7/64; A61K 38/00; C07K 2319/70

(57) ABSTRACT

A small peptide drug with AR protein degrading capacity for prostate cancer-specific treatment.

### FIELD

This patent application relates to a novel anti-cancer therapy for prostate cancer based on small peptide technology.

### BACKGROUND

Prostate cancer is a significant health issue in Western countries, and it is the second leading cause of cancer death in American men, behind only lung cancer [1]. About one man in 8 will be diagnosed with prostate cancer during his lifetime, and about one man in 41 will die of prostate cancer in the US, according to the ACS website description (www.cancer.org). It is estimated that 268, 490 new cases will be diagnosed, and 34,500 prostate cancer-related deaths will occur in US men this year [1]. Although the 5-year survival rate for localized diseases is about 100%, this rate drops to 30% for metastatic diseases [2].

Currently, metastatic prostate cancers are initially managed with androgen deprivation therapy (ADT) [2] because prostate cancer depends on the androgen receptor (AR), a transcriptional factor critical for prostate cancer growth and progression [3]. Castration by surgery or medical treatment reduces androgen levels, resulting in prostatic atrophy and prostate cancer regression. However, metastatic prostate cancers often relapse and progress to a stage termed castration-resistant prostate cancers (CRPC) after initial androgen deprivation therapy [4]. Clinical treatments for CRPC are focused on suppressing AR activity with antagonists like Enzalutamide/Apalutamide or reducing androgen production from the adrenal gland and prostate cancer cells with Abiraterone [5]. However, these treatments fail to yield a meaningful benefit in CRPC patients due to AR gene mutations or splice variations (i.e., AR-v7/v12) [3, 6, 7], or cross-activation by other cellular signal pathways, resulting in AR re-activation [8]. Therefore, a different strategy is urgently needed to shut down the persistent AR signaling activity. Based on previous studies from my research group [9-11] and other groups [12-18], it is conceivable that reducing the AR gene expression or eliminating the AR protein in prostate cancer cells is a feasible and attractive solution to provide a curative outcome for castration-resistant prostate cancers [3].

In a very recent article published in March 2022 [3], my collaborators and I described the current status of research efforts in eliminating the AR signaling activity in prostate cancer in cell culture and animal xenograft models. The small interference RNA [9-11] and antisense oligonucleotide [12-18] technologies were used to reduce AR gene expression at the mRNA level. Due to the difficulties of intracellular delivery of the therapeutic nucleotide molecules, our group stalled the clinical development of these technologies several years ago. On the other hand, recent studies showed that several small chemical compounds were developed to enhance AR protein degradation in prostate cancer cells. The most popular technology was the PROTAC molecules, which use substrate-specific ligands to bring together the AR protein with a ubiquitin ligase E3, resulting in proteasome-mediated AR protein degradation. Since the ligands used in the PROTAC molecules were AR c-terminal ligand binding domain-specific, these ligands were not functional toward the AR slicing variants like AR-V7, leading to treatment failure [20-22]. Other small molecules were not AR protein-specific, so the efficacy was in question, as summarized in our review article [3].

### SUMMARY

The technology was to use an 8-mer (termed as ARi8) small peptide to trigger the AR protein degradation in prostate cancer cells as a novel treatment for prostate cancer patients. The peptide is fused with a PSMA-binding GTI peptide at its N-terminal for prostate cancer cell-specific delivery. The ARi8 peptides are demonstrated to induce AR protein degradation encoded by the mutant (FIG. 1B), wild-type AR gene (FIG. 2B), or spliced AR gene (FIG. 4C). Sustained AR protein degradation led to apoptotic cell death in prostate cancer cells (FIG. 2C & 3B) and reduced xenograft tumor growth of prostate cancer models in castrated nude mice (FIG. 7-8). Replacement of the ARi8 peptide sequence at S, R, D, and N residues with T, H, E, and G amino acids, respectively, retained the AR protein degrading activity (FIG. 5). Replacing the alpha-amino acid arginine (ARG, R) residue with beta-amino acid residue (beta-homo-R) also retained the AR degrading activity (ARil0alpha/alpha/beta, FIG. 6). Cyclic. linear peptide also enhanced the efficiency of the AR degradation (FIG. 9). ARi8 analogs (ARi8.3, ARi8.4, ARi10alpha/alpha/beta) and their cyclic analogs are candidate drugs for prostate cancer treatment.

### BRIEF DESCRIPTION OF THE DRAWINGS & FIGURES

FIG. 1. ARi14 reduces AR tyrosine phosphorylation and AR protein levels.

A. LNCaP cells were serum-starved overnight and then treated with R1881 (1.0 nano-M) with or without myrARi14 (10 micro-M) for 4 h in 2% charcoal-stripped FBS-supplied media. Protein lysates were used for anti-AR (clone PG21) immunoprecipitation, followed by western blot with anti-phospho-tyrosine antibody (clone 4G10). The membrane was then re-probed with an anti-AR antibody for protein loading control. B. LNCaP cells were serum-starved overnight and then treated with myrARi14 at different concentrations (3 or 10 micro-M for 24 h) or periods (16-24 h). Actin blots served as a protein loading control.

FIG. 2. ARi14 induces AR protein degradation and cell death in prostate cancer cells.

A. ARi14 peptide was fused with the KDM4A CPP sequence via a short link (KDM4A-ARi14). This fused peptide was labeled with FITC fluorescent dye. B. LAPC-4 cells were treated with the solvent, or the peptides as indicated at 10 micro-M in 2% charcoal-striped FBS-supplied media for 24 h. Cellular proteins were subjected to a western blot with an anti-AR antibody (clone PG21). Actin blot served as a protein loading control. C. Microscopic images were shown for cell viability. Note: white arrows indicate a cluster of dying cells loaded with fluorescent-labeled peptides.

FIG. 3. ARi8 peptide maintains the AR degrading activity.

A. C4-2B cells were treated with peptides (10 micro-M) for 24 h as indicated, and AR protein levels were assessed by western blots. B. C4-2B cells were treated with peptides and harvested for western blot assay with an anti-PARP antibody. Actin blot served as a protein loading control. C. Flow cytometry analysis of subG1/GO cell cycle population in C4-2B cells after peptide treatment (10 micro-M) in 2% charcoal-stripped FBS for 24 h. Data represents two experiments. The asterisk indicates a significant difference compared to the DSMO control (p<0.05, Student t-test).

FIG. 4. GTI-ARi8 specifically targets PSMA-positive cells and reduces AR protein levels.

A&B. C4-2B and PC-3 cells were cultured in serum-free media, and FITC-conjugated GTI-ARi8 peptides were added to the cell culture at different concentrations (5, 10, and 20 micro-M) overnight. C. 22RVI cells were treated with GTI-ARi8 (5-10 micro-M) or scramble control (10 micro-M) for 24 h in 5% charcoal-stripped FBS. Western blot assays were conducted for AR level (clone PG21 antibody). Actin blot served as a protein loading control.

FIG. 5. The ARi8 analogs evaluation.

A. The ARi8 sequence was substituted on 4 residues (green font). Peptides were synthesized as N-terminal myristylation signal modification. The ARi8-s was a negative control peptide with a scrambled sequence without a known human protein match. B. LNCaP cells were treated with peptides as indicated for 24 h, and cellular protein lysate was used for the anti-AR (clone PG21) immunoblot assay. Actin blot served as a protein loading control. C. Quantitative comparison of the AR protein band density was summarized after normalization with Actin blot.

FIG. 6. ARi10alpha/alpha/beta peptide induces AR protein degradation in prostate cancer cells.

A. Peptide analogs were synthesized as listed in the top panel. The myristylation sequence was conjugated to the N-terminal of the peptide for penetrating the plasma membrane. B. Prostate cancer C4-2B cells were treated with the agents at a concentration of 10 micro-M for 48 h. Cells were harvested for western blot assay with AR antibody (clone PG21). The Actin blot served as a protein loading control.

FIG. 7. GTI-ARi8 suppresses CRPC xenograft tumor growth.

Subcutaneous xenograft tumors were established in castrated nude mice using 22RV1 (panel A) and C4-2B cells (panel B). Once tumor mass reached 3 mm in diameter, animals were treated with peptides by s.c. injection on the opposite side of the tumor mass at a dose of 5.0 mg/kg body weight twice a week for two weeks. Tumor growth was monitored by measuring the diameters with a caliper. The asterisk indicates a significant difference between the two groups (p<0.05, Student t-test). Inserted panels: anti-AR (clone AR441) immunohistochemistry staining.

FIG. 8. GTI-ARi8.4 peptides suppressed xenograft tumor growth derived from prostate cancer cells. C4-2B cells (5x 106) were used to establish the subcutaneous xenograft tumors in 6-week-old nude male mice after castration. Intraperitoneal injection (three times a week for three weeks) with the solvent or GTI-ARi8.4 peptides (5-10 mg/kg body weight) was started one week after cancer cell inoculation. A. Tumor growth was monitored using a digital caliper. The asterisk indicates a significant difference between the two groups (ANOVA analysis, * p<0.05; ** p<0.01). B. Animal body weight was recorded simultaneously. C. Xenograft tumors were harvested at the end of treatment for a macro photo.

FIG. 9. Cyclic GTI-ARi8 peptide design and evaluation for AR degradation in prostate cancer cells.

A. GTI-ARi8 peptide was cyclically stapled using two inserted cysteine residues at positions 5 and 26 with 4,4′-Bis (bromomethyl) Biphenyl, as indicated by the asterisk. B. Schematic illustration of cyclic GTI-ARi8 in a 3-D model generated by PEP-FOLD3 (https://bioserv. rpbs. univ-paris-diderot.fr/services/PEP-FOLD3/). The text colors of the peptide chain are corresponsive to the fragments in a 3-D structure. C. The cyclic GTI-ARi8 peptides induced AR degradation in prostate cancer C4-2B and 22RV1 cells, which is much more potent than the linear GTI-ARi8 peptide.

### DETAILED DESCRIPTION

In the following section, the small peptide technology according to the teachings for this application in the form of a therapeutic drug for prostate cancers will be described by the embodiments. It should be noted that although only prostate cancers are targeted for this treatment, the teachings of this application can also be used in any other types of human cancers harboring the active AR protein as a driving force of disease progression, such as liver cancers, bladder cancers, as reviewed in the literature [23, 24].

This section describes the development of small peptide-based novel therapeutic drugs for castration-resistant prostate cancer (CRPC) treatment. These peptides triggered AR protein degradation, specifically in prostate cancer cells. Our results showed that these peptides eliminated AR proteins (full-length, mutant, or truncated variants) in prostate cancer cells, resulting in tumor retardation.

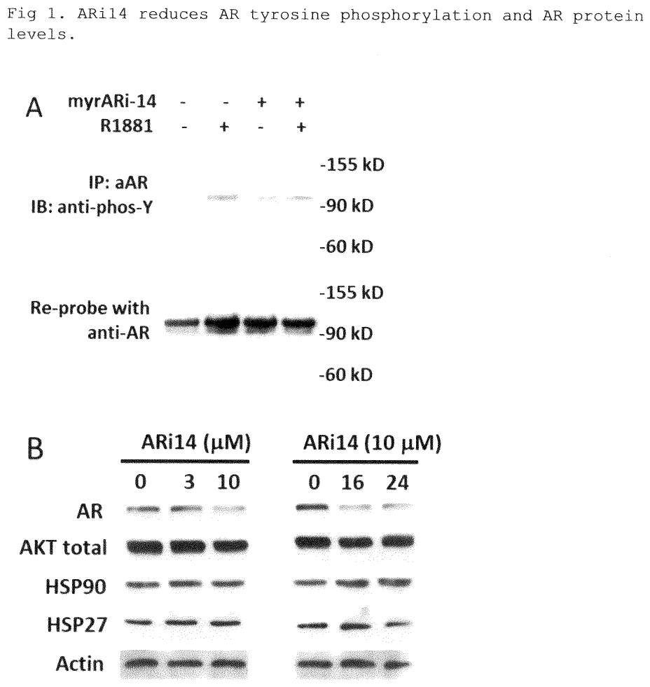

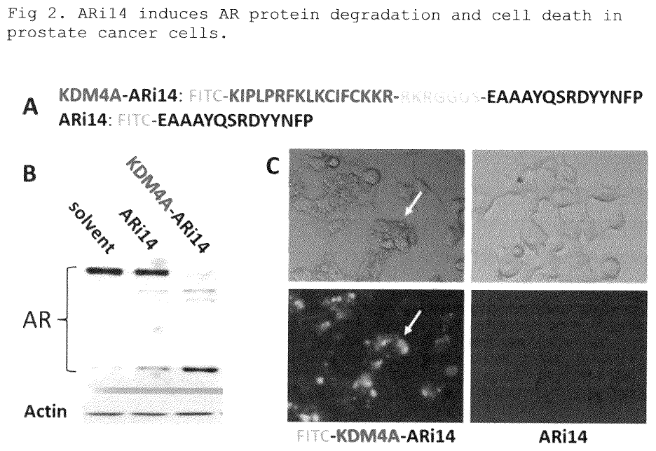

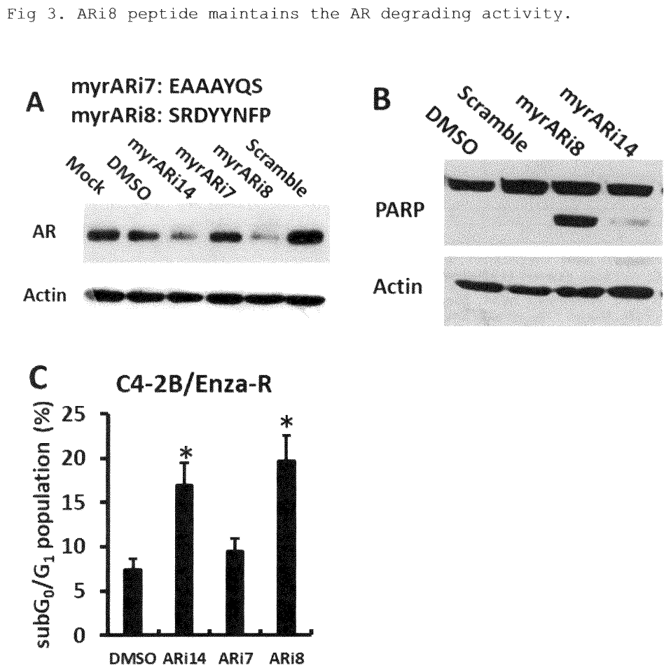

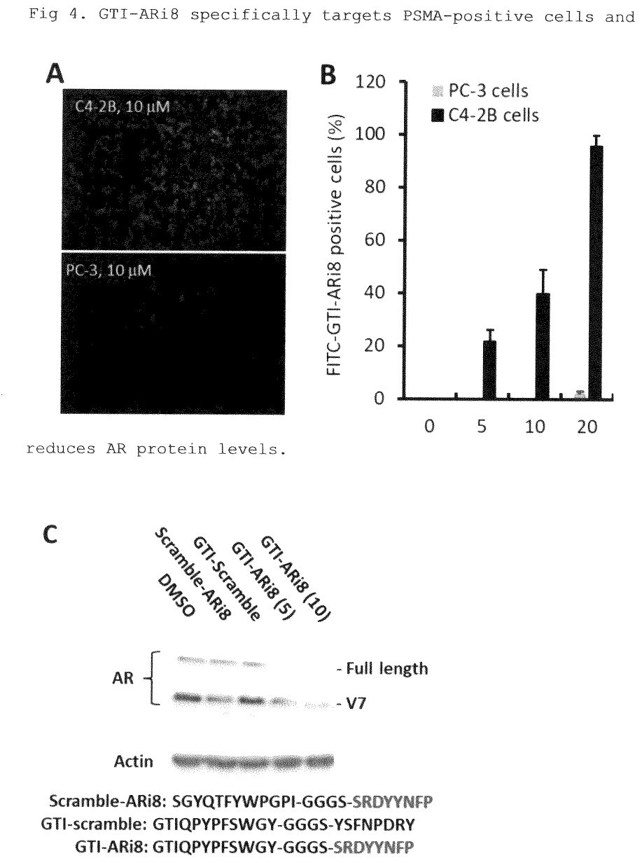

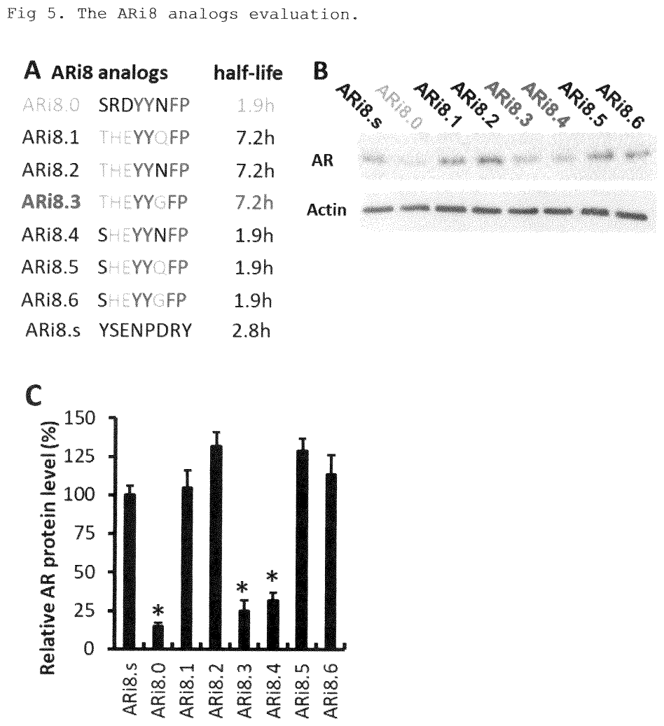

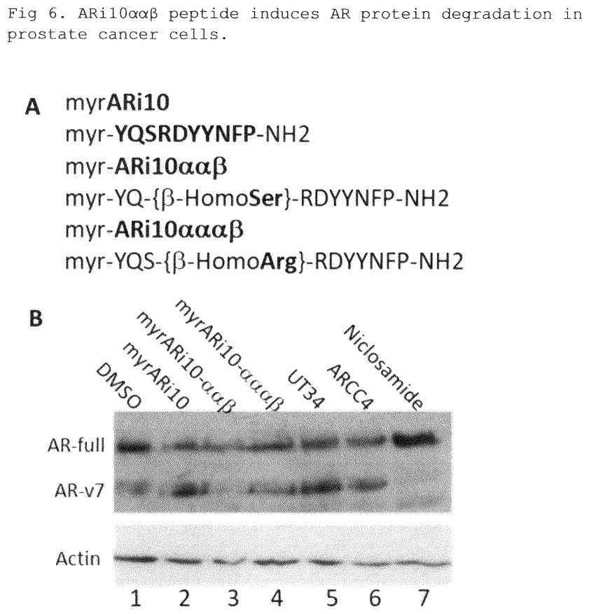

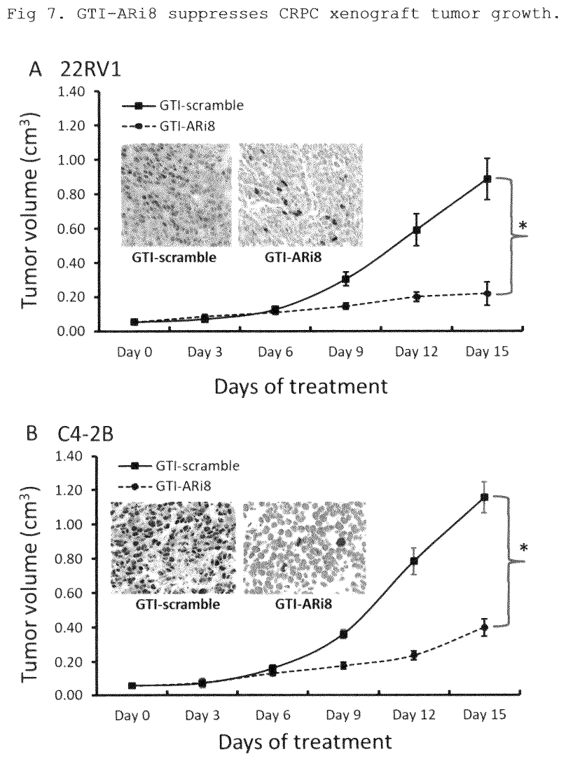

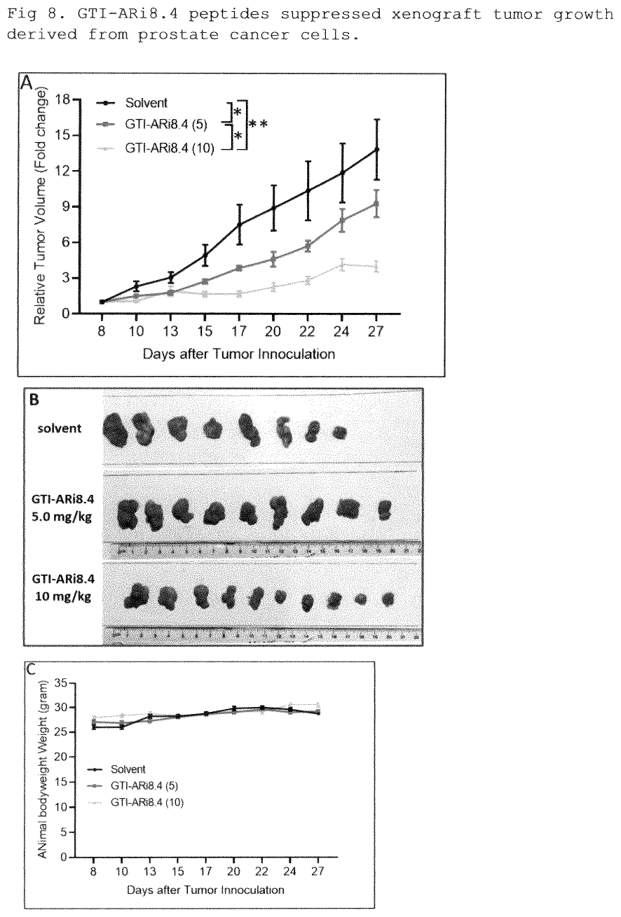

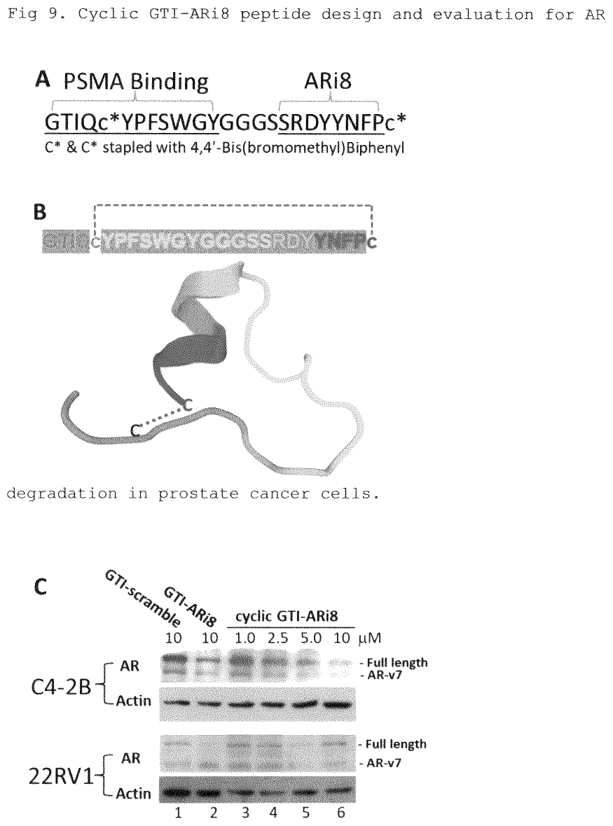

The small peptide was initially designed as a decoy molecule derived from the AR N-terminal domain (amino acid sequence 355-EAAAYQSRDYYNFP-368, ARi14) to black AR N-terminal tyrosine phosphorylation. This design was inspired by two studies showing AR N-terminal tyrosine phosphorylation by tyrosine kinases Src and Ack1, which are critical for AR activation and CRPC progression [25-30]. Within the ARi14 peptide sequence, there is a cluster of three tyrosine residues (Y359/Y364-365). We first tested the ARi14 effect on AR tyrosine phosphorylation in androgen-responsive prostate cancer LNCaP cells. To facilitate the penetration of the ARi14 peptide crossing the plasma membrane, we conjugated the ARi14 peptide with a myristoylation signal at the N-terminal for living cell uptake [31, 32], named myrARi14. As expected, androgen (R1881) stimulation for 4 h in LNCaP cells induced a substantial increase in the total AR tyrosine phosphorylation level, which was partially reduced after myrARi14 co-treatment with R1881 (FIG. 1A). Surprisingly, a more extended treatment period with myrARi14 peptide drastically reduced AR protein levels (FIG. 1B). However, the AR gene expression at the mRNA level by qPCR assay did not attenuate after myrARi14 treatment (negative data did not show), indicating a post-translational degradation of the AR protein.

Then, we used a secondary approach to confirm the ARi14 peptide-induced AR protein degradation. To achieve a better intracellular distribution of the peptide, a cellular penetrating peptide (CPP) derived from the KDM4A protein was fused to the N-terminal of the ARil4 peptide via a short link sequence GGGS (FIG. 2A). This fusion peptide drastically reduced AR protein level and induced a profound cell death response in the androgen-sensitive prostate cancer LAPC-4 cell line. At the same time, the non-conjugated ARi14 had no apparent effect on AR protein levels or cell viability (FIG. 2B & 2C).

We further characterized the ARi14 peptide for its core sequence of action. The ARi14 peptide was chopped into two fragments, the 7-mer ARi7 peptide (EAAAYQS) and 8-mer ARi8 peptide (SRDYYNFP), and their AR degradation inducing ability was compared to the original 14-mer peptide (FIG. 3). These peptides were synthesized with an N-terminal myristylation signal to facilitate cellular uptake. As shown in FIG. 3A, ARi7 showed no impact on the AR protein level. Surprisingly, ARi8 exerted a much stronger effect on reducing AR protein level than the original ARil4 peptide in CRPC C4-2B cells (92% AR reduction by ARi8 vs. 78% reduction by ARil4). ARi8 also induced more profound apoptotic cell death than ARi14, as evidenced by PARP cleavage (FIG. 3B). Most interestingly, both ARi8 and ARi14 induced cell cycle arrest at the subG1/GO phase in Enzalutamide-resistant C4-2B cells (FIG. 3C).

To achieve a prostate cancer-specific delivery, we used a prostate-specific membrane antigen (PSMA) binding peptide (GTI peptide), which was identified as highly affinitive and specific to prostate cancer cells [35]. We fused this GTI peptide with the ARi8 peptide via a short GGGS linker [36]. We tested the prostate-specific ARi8 fusing peptide (GTI-ARi8) in AR/PSMA-positive CRPC C4-2B cells and PSMA-negative PC-3 cells. Our data confirmed the GTI-ARi8 specificity in PSMA-positive C4-2B cells but not in PSMA-negative PC-3 cells (FIG. 4A & 4B). We also tested GTI-ARi8 in 22RV1 cells expressing full-length AR and AR-V7 variants. As shown in FIG. 4C, the GTI-ARi8 fusion peptide remarkably reduced AR protein levels (full-length AR and AR-V7), confirming the effectiveness of the peptide on AR degradation.

To fine-tune the peptide composition, we made several substitutions on the ARi8 peptide by replacing the S, R, D, and N residues (position 1-2-3---6) with the amino acids of T, H, E, and Q/G, respectively, that are in the same sub-categories of polarity or side chain charge (FIG. 5A). As shown in FIG. 5B & 5C), the analogs ARi8.3 and ARi8.4 retained a similar AR-reducing effect as the original ARi8.0 peptide. However, the half-life of ARi8.3 increased dramatically to 7.2 h (compared to ARi8.0, 1.9 h), as predicted by the ExPASy ProtParam program. The ARi8.3 and ARi8.4 will be further developed as clinical drugs.

Despite numerous advances that emerged in peptide therapeutics with efficacious, relatively safe, preferred tolerability, and a wide range of disease applications, natural alpha-amino acid peptides are not preferred drug candidates because of their high proteolytic susceptibility, low conformational stability, and poor plasma membrane permeability [23-25]. Replacing natural alpha-amino acid residues with unnatural beta-amino acid residues in a single chain leads to heterogeneous backbone oligomers called alpha/beta-peptides [26, 27]. Despite their unnatural backbones, alpha/beta-peptides were found to manifest various folding patterns and biological functions reminiscent of natural peptides and proteins with improved stability [27, 28]. So far, alpha/beta-peptides have been developed to target diverse biological targets, including apoptotic signaling, HIV-cell fusion, hormone signaling, and angiogenesis in vitro and in vivo [23, 24, 26, 29]. To improve the drug-like property of the ARi8 peptides, we replaced the third and fourth alpha-amino acid residues with a homo beta-amino acid based on the model described in a previous report [28]. As shown in FIG. 6, a new analog ARi10 was synthesized and the alpha/beta-peptides with the beta-substitution at the 3rd/4th position were denoted as ARi10alpha/alpha/beta and ARi10 alpha/alpha/alpha/beta (FIG. 6A). Treatment of prostate cancer C4-2B cells with these peptides largely reduced AR protein levels (lane 2-4 vs. lane 1), especially for the ARil0alpha/alpha/beta peptide with the highest potency (lane 3), compared to other peptides and PROTAC agents ARCC4 and UT34 (FIG. 6B). As reported previously, the anti-warm drug Niclosamide induced AR-v7 degradation [19]. These data proved that beta-amino acid substitution on AR degradation-inducing peptide is a feasible approach to developing a drug-like peptide agent for AR degradation in prostate cancers.

Next, we tested these peptides in animal experiments. We established subcutaneous xenografts with 22RV1 and C4-2B cells in nude mice. GTI-ARi8 and the scrambled peptide were dissolved with 20% DMSO in PBS solution and were delivered via subcutaneous injection. As shown in FIG. 7, compared to the scrambled peptide, the GTI-ARi8 peptide significantly reduced tumor growth (tumor volume) and AR protein expression in the xenograft models.

We then tested the anti-tumor effect of the modified ARi8.4 peptide in animal xenograft models. The peptides were synthesized as the GTI-fused peptide for prostate cancer-specific delivery. The peptides were dissolved in an acidic condition (3% Acetic acid in PBS) and then neutralized with sodium hydroxide. Prostate cancer C4-2B cells were used to establish the xenografts in nude mice as described [11]. The GTI-ARi8.4 peptides were used at two doses (5 & 10 mg/kg body weight) three times a week for three weeks by intraperitoneal injection. As shown in FIG. 8A & 8B, GTI-ARi8.4 treatment significantly suppressed xenograft tumor growth in a dose-dependent manner. However, animal body weight had no significant difference between the solvent and treatment groups (FIG. 8C), indicating low toxicity of the peptides.

Currently, more than 65 peptides have been approved and used as therapeutics to treat patients and about 150 peptides are being evaluated in different phases of clinical trials [37]. Integrilin and Octreotide are two marketed cyclic peptides that have been used to treat thrombosis and cancer, respectively [37]. Some previous problems of peptide stability in plasma and formulation have been overcome using various methods including cyclization [38]. Cyclic peptides have been shown to improve the plasma and chemical stability of peptides and cellular uptake [39]. In addition, the formation of a cyclic peptide has been shown to induce backbone conformational restriction to improve the peptide's binding selectivity for the target protein such as a receptor or enzyme. To enhance the drug-like property, we designed a cyclic GTI-ARi8 peptide as shown in FIG. 9A & 9B. Our cell-based testing results showed that the cyclic GTI-ARi8 treatment largely reduced AR protein levels in a concentration-dependent manner both in C4-2B and 22RV1 cells (FIG. 9C). Meanwhile, the cyclic GTI-ARi8 was much more potent than the linear GTI-ARi8 peptide (lane 6 vs lane 2 in C4-2B cells and lane 5 vs lane 2 in 22RV1 cells, FIG. 9C). These data indicated that the cyclic GTI-ARi8 is a much better AR degradation inducer.

The Advantages of AR Degrading Peptides Vs. Other Similar Agents:

The small peptide technology described in this application possesses several advantages over other compounds and existing clinical therapies. First, the small peptide technology is specific for the AR protein degradation; second, the peptide could be synthesized using B-amino acid as a substitution for the arginine residue at position-2, a critical modification for avoiding peptidase digestion. This client protein-targeted peptidase-resistant (ARi8.3, ARi8.4, and ARi10 alpha/alpha/beta) peptides will be developed as a potent therapy for advanced prostate cancers.

Targeting AR protein stability has emerged as the hotspot in developing new therapeutics for advanced prostate cancers in recent years. Several small molecules were reported to reduce AR protein stability in prostate cancers [3]. For example, the curcumin analog ASC-J9, anti-worm drug Niclosamide, AR-NTD inhibitor UT-34, and other small molecules induced AR protein degradation in prostate cancer cells [3]. However, these small molecules have not established AR-specific targeting and prostate cancer tissue specificity [3].

In addition, the recently developed PROTAC technique for AR degradation depends on an existing AR ligand. An intact AR C-terminal ligand-binding domain (AR LBD) is required to be effective [3]. However, as CRPC patients often express a mutant or truncated protein that lacks the ligand-binding domain like AR-V7, the PROTAC technology will suffer from drug resistance in these CRPC patients because the AR-binding moieties on the PROTAC molecule will miss the AR variant protein like AR-v7. One exception is the MTX-23 PROTAC agent, which uses an AR DNA binding domain (AR DBD) inhibitor as the targeting ligand. Despite this elegant design with the AR DBD targeting that bypasses the defect in the first generation of AR PROTAC with AR LBD binding, the off-target potential on other DNA binding proteins is not yet ruled out.

Currently, there are several AR N-terminal targeted small molecules (EPI compounds) that suppress AR transactivation [3]. However, these EPI compounds alone or combined with AR antagonists or radiation did not affect AR protein degradation in prostate cancer cells or xenograft tissues. In addition, the first-generation EPI compound EPI002/Ralaniten failed in a clinical trial (NCT02606123) due to the extreme bill burden.

This failure is possibly related to the lack of AR protein degradation compared to our small peptide technology.

Although short peptides were previously used to interfere with the interaction between AR and its co-activators, only a partial suppression in AR transactivation was observed without AR protein reduction [3]. Our small peptide technology will be the first-in-class therapeutic peptide that eliminates AR protein by disturbing AR protein stability through a unique mechanism (data not published yet).

Terms Used in this Patent Invention:

As used herein, the term “amino acid” includes naturally occurring amino acids and synthetic amino acids, as well as amino acid analogs and amino acid mimetics that function like naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refer to compounds with the same basic chemical structure as a naturally-occurring amino acid, e.g., an alpha-carbon bound to a carboxyl group, an amino group, and an R group, e.g., homoserine, norleucine, methionine sulfoxide, methionine methyl sulfonium. Such analogs may have modified R groups (e.g., norleucine) or modified peptide backbones but retain the same basic chemical structure as a naturally occurring amino acid. Amino acid mimetics refer to chemical compounds with a structure that is different from the general chemical structure of amino acids but that functions like a naturally occurring amino acid. Amino acids can be referred to herein by either their commonly known three-letter symbols or by the one-letter symbols recommended by the IUPAC-IUB Biochemical Nomenclature Commission.

1. A peptide that is
SRDYYNFP (“ARi8”), or
THEYYGFP (“ARi8.3”), or
SHEYYNFP (“AR18.4”), or
YQSRDYYNFP (“ARi10”), or
YQ-[beta-homo-S]-RDYYNFP (“ARi10 alpha/alpha/beta”), or
YQS-[beta-homo-R]-DYYNFP (“ARi10 alpha/alpha/beta”), or
a pharmaceutically acceptable salt and/or a solvate thereof.

2. A peptide of claim 1, optionally where one or more amino acids are substituted with D-type amino acids, or beta-homo amino acids.

3. A fusion peptide of claims 1 and 2, wherein R1, R2, R3, R4, R5, and R6 are each independently a targeting peptide or small molecules for cell type-specific targeting peptide or small molecules
R1-SRDYYNFP,
R2-THEYYGFP,
R3-SHEYYNFP,
R4-YQSRDYYNFP,
R5-YQ-[beta-homo-S]-RDYYNFP,
R6-YQS-[beta-homo-R]-DYYNFP,
or a pharmacologically acceptable salt and/or solvate thereof.

4. A cyclic peptide of claims 1-3, where the peptides are stapled for cyclization, and a pharmacologically acceptable carrier and/or a pharmacologically acceptable excipient.

5. A composition comprising a peptide of claims 1-4; and a pharmacologically acceptable carrier and/or a pharmacologically acceptable excipient.

6. A method comprising administering a fusion peptide of claim 1-5 to a subject suffering from prostate cancer.
